(19) United States
(12) Patent Application Publication
(10) Pub. No.: US 2026/0351381 A1
(43) Pub. Date: Nov. 5, 2026

(54) Perioperative Cobotic System with Multiple Imaging Modalities for Environmental Awareness

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/432,236

(22) Filed: Dec. 24, 2025

Related U.S. Application Data
(63) Continuation-in-part of application No. 19/195,731, filed on Apr. 30, 2025.

(51) Int. Cl. G16H 20/40; G16H 30/20

(52) U.S. Cl. CPC G16H 20/40; G16H 30/20

(57) ABSTRACT

A perioperative cobotic system is disclosed for intraoperative environmental awareness and task execution. The system includes a humanoid mobile cobot equipped with a multi-modal imaging suite comprising RGB cameras, depth sensors, thermal imagers, and panoramic lenses. Sensor data are fused to generate a unified, real-time spatial map of the operating room, including dynamic personnel, equipment, and sterile field boundaries. A surgical phase detection engine applies machine learning to infer procedural stages from visual patterns. A context-aware task execution system triggers cobot actions aligned with surgical workflow events, while a human interaction interface supports gesture and voice-based commands. The system adapts to lighting changes and occlusions using redundant sensing and dynamic fusion logic. Logged imaging and system data support real-time visualization and postoperative review. Safety and privacy controls enforce sterile field compliance and data access restrictions. The system enables autonomous, responsive support for surgical teams during complex intraoperative procedures.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to robotic systems in surgical environments and, more particularly, to perioperative cobotic platforms equipped with multi-modal imaging and real-time mapping capabilities for environmental awareness, workflow adaptation, and intraoperative collaboration.

### BACKGROUND

Modern operating rooms are complex, high-stakes environments in which surgical teams must maintain sterility, coordinate across multiple roles, and dynamically respond to evolving procedural demands. In this context, perioperative cobots, collaborative robotic platforms designed to work alongside human teams, have emerged as promising tools for improving surgical efficiency, reducing clinician workload, and enhancing consistency in task execution. However, current cobotic systems remain limited in their ability to perceive and interpret their surroundings with the granularity and adaptability required for real-time intraoperative support.

Conventional robotic systems in surgical settings typically rely on basic imaging modalities, such as fixed optical cameras or low-resolution depth sensors, for obstacle avoidance and positioning. These sensors, while sufficient for coarse navigation, offer limited spatial resolution, field of view, or adaptability to changing environmental conditions. They are often incapable of distinguishing between sterile and non-sterile zones, detecting subtle workflow cues, or responding appropriately to occlusions, shadows, or equipment movement during active procedures. Furthermore, many existing platforms do not integrate multiple imaging sources or perform sensor fusion to form a unified understanding of the operative space.

The absence of a real-time, high-fidelity spatial model also restricts cobots from meaningfully engaging with dynamic intraoperative workflows. Without comprehensive mapping, cobots cannot anticipate changes in team positioning, interpret surgical phases, or adjust behaviors based on procedural context. Additionally, current systems typically lack the software intelligence necessary to detect surgical phases, recognize gestures or verbal cues from surgeons, or modulate their activity based on lighting or field occlusion. As a result, most intraoperative cobots operate in reactive or semi-autonomous modes, with minimal understanding of their role in the broader procedural timeline.

Moreover, safety, privacy, and compliance concerns impose further constraints. Cobotic systems operating in perioperative settings must ensure compliance with sterile field integrity, hospital privacy policies, and regulatory requirements such as HIPAA. These constraints are difficult to meet without fine-grained environmental perception and role-based access controls for visual data.

There remains a need for an advanced perioperative cobotic system capable of autonomously operating in intraoperative environments through integration of multiple imaging modalities, real-time sensor fusion, spatial mapping, and intelligent context recognition. In particular, there is a need for systems that can dynamically adapt to procedural phases, respond to human cues, maintain visual continuity despite environmental variability, and operate in compliance with safety and privacy protocols. The present invention addresses these and other deficiencies in existing perioperative robotic platforms.

### SUMMARY

The present disclosure relates to a perioperative cobotic system equipped with multiple imaging modalities for real-time environmental awareness and intraoperative task adaptation. The system includes a mobile humanoid cobot platform designed to autonomously navigate surgical environments, including preoperative, intraoperative, and postoperative zones, while maintaining sterile-field compliance and continuous situational awareness.

A multi-modal imaging suite is integrated into the cobot platform, comprising optical RGB cameras for visual tracking of personnel and tools, depth sensors or LiDAR for real-time three-dimensional mapping, thermal imaging units for heat-based detection of human presence and equipment status, and ultrawide or fisheye cameras for panoramic awareness and blind spot coverage. These imaging modalities collectively provide comprehensive environmental perception under variable lighting, occlusion, and spatial conditions.

Sensor data from the imaging suite are processed by a sensor fusion and environment mapping module that generates a unified, continuously updated three-dimensional map of the operating room. This map identifies static objects, dynamic obstacles, team members, and sterile field boundaries, enabling the cobot to position itself safely and adapt its behavior in real time.

The system further includes a context-aware task execution engine that interprets visual and spatial inputs to autonomously trigger or adjust cobotic actions based on observed workflow events. For example, the cobot may reposition itself in response to line-of-sight constraints, prepare for an instrument handoff, or initiate a support task in coordination with team activity.

A surgical phase detection engine employs machine learning models trained on annotated surgical procedures to infer the current procedural phase, such as setup, incision, or closure, from visual patterns and team behavior. Based on the identified phase, the cobot can execute anticipatory actions such as pre-positioning sensors, adjusting visual perspective, or preparing task modules relevant to the upcoming workflow stage.

The system further supports collaborative human interaction through visual and auditory interfaces capable of recognizing gestures and voice commands from surgical team members. Lighting and occlusion compensation modules adaptively modulate imaging parameters to maintain consistent visual input despite shadows, drapes, or personnel movement.

A logging and visualization subsystem records synchronized multi-modal imaging data, cobot behavior, and procedural context for postoperative review, compliance tracking, and machine learning refinement. Real-time visualization tools enable the surgical team to view the cobot’s spatial awareness map and status on local or remote displays.

The system includes built-in safety, privacy, and compliance features such as collision detection, role-based access to visual data, and regulatory safeguards for HIPAA compliance and sterile field integrity. The invention thus provides an integrated cobotic solution for autonomous support in intraoperative workflows, combining rich environmental perception, intelligent context interpretation, and responsive collaborative behavior.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of a perioperative cobotic system with a mobile base and integrated multi-modal imaging suite.

FIG. 2 is a block diagram of the system architecture.

FIG. 3 is a block diagram of the multi-modal imaging inputs.

FIG. 4 is a diagram of the sensor fusion and environment mapping module.

FIG. 5 is a process diagram illustrating the operation of the surgical phase detection engine.

FIG. 6 is a flowchart of the context-aware task execution system.

FIG. 7 is a block diagram of the human detection and interaction interface.

FIG. 8 is a schematic representation of the lighting and occlusion compensation logic.

FIG. 9 is a diagram showing the real-time visualization and logging subsystem.

FIG. 10 is a flowchart of the safety, privacy, and compliance control pipeline.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

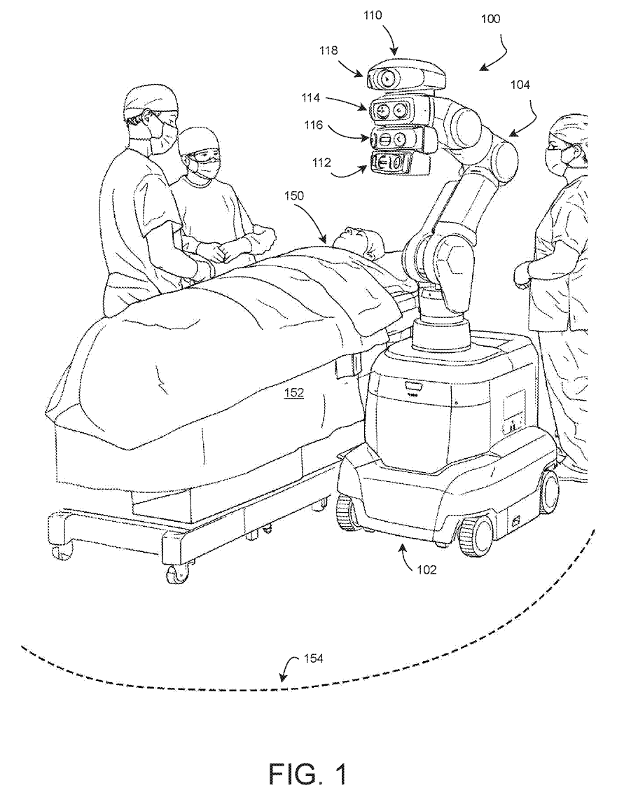

Embodiments of the present invention are described below with reference to the accompanying FIGS. 1 through 10, which illustrate exemplary implementations of a perioperative cobotic system with multiple imaging modalities for environmental awareness. These examples are provided in sufficient detail to enable those skilled in the art to practice the invention, but are not intended to be limiting. It will be understood that various changes and modifications may be made to the system components and workflow without departing from the scope of the invention.

In the figures, like reference numerals refer to like components throughout the drawings. For clarity of illustration, certain elements may be shown in a simplified form or exaggerated in scale, and not all components may be depicted in every figure.

Referring to FIG. 1, a perspective view of an exemplary perioperative collaborative robotic (cobotic) system 100 is shown in an operating room (OR) environment. The cobot platform 100 includes a mobile base 102 supporting an articulated robotic arm 104. The base 102 houses drive motors and wheels (e.g., an omnidirectional or differential drive system) that enable autonomous navigation around the OR floor. In this embodiment, the base 102 also contains an onboard power source (such as a battery) and a control unit (computer processor and memory) for real-time operation. The platform 100 is dimensioned and constructed for use in sterile or near-sterile environments, with smooth, easy-to-clean surfaces and sealed components to comply with hospital hygiene protocols.

The robotic arm 104 extends from the base and is configured to position sensors and tools within the surgical environment. Arm 104 may have multiple degrees of freedom (e.g., a 6-DOF articulated arm) to allow precise movement and positioning. In some embodiments, arm 104 can hold and manipulate surgical instruments or accessories (for example, to pass a tool, adjust a sterile drape, or operate a suction device) under the control of the cobot. The arm and base are equipped with force-limited actuators and collision detection sensors so that the system can safely operate in close proximity to surgical staff and patients. The platform 100 features rounded edges, protective covers, and other safety design elements to avoid snagging drapes or equipment and to prevent injury upon contact with personnel.

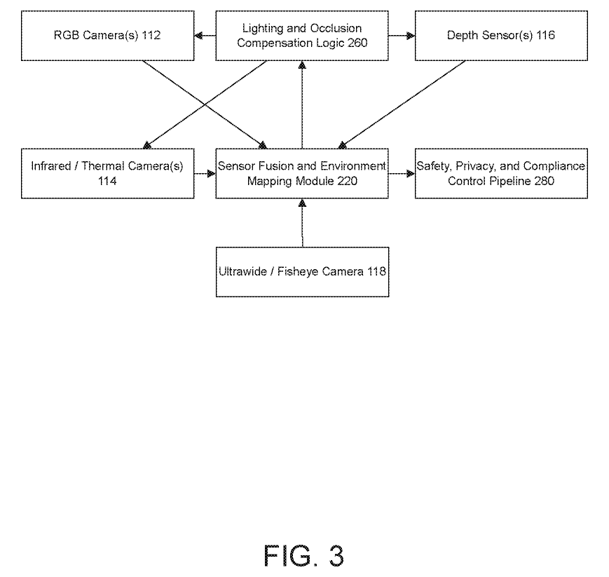

Mounted on the cobot 100 is a multimodal imaging suite 110 comprising various imaging and depth-sensing devices (detailed further in FIG. 3). One or more high-resolution RGB color cameras 112 are positioned on the arm 104 or at strategic locations on the base 102 to capture visual details of the surgical field, staff, and equipment. Thermal or infrared cameras 114 are included to detect heat signatures of people and devices, aiding in human presence detection and equipment status monitoring even in low-light conditions. Additionally, depth sensors 116 (for example, a LiDAR unit or stereo camera pair) are integrated to provide three-dimensional spatial perception of the OR. An ultrawide-angle or fisheye camera 118 may be mounted high on the cobot (for instance, atop the arm 104 or on a mast) to give panoramic awareness of the room and eliminate blind spots. Each sensor in the imaging suite 110 is operatively coupled to the control unit in base 102, supplying continuous data streams for analysis.

FIG. 1 illustrates the cobot 100 positioned near an operating table 152 on which a patient 150 lies, with the surgical team (e.g., a surgeon and nurses) standing nearby. The cobot 100 can autonomously navigate to an optimal location in the OR, for example at the periphery of the sterile field 154, to observe the procedure without interfering. The system 100 is designed with sterile-field compliance: it maintains a set distance from the sterile surgical field 154 unless its task explicitly requires close approach. If the arm 104 or any component needs to extend toward a sterile area, the system can be fitted with sterile drape covers or a sterilizable sleeve to preserve sterility. During operation, platform 100 remains aware of its surroundings and can reposition itself as needed (via movement of base 102 or adjustment of arm 104) to maintain line-of-sight to key activities or to yield to human movement, all while keeping critical areas unobstructed.

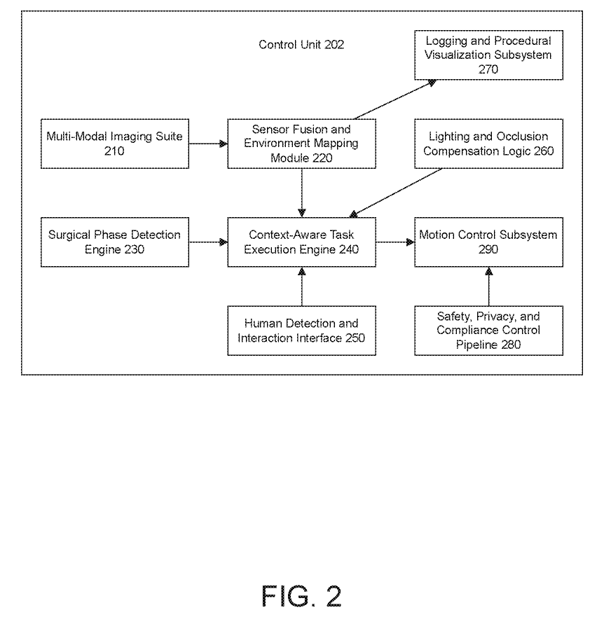

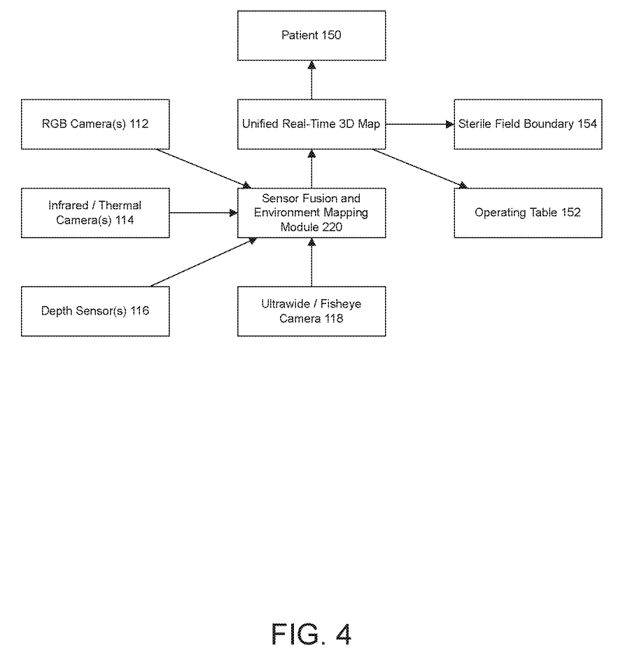

FIG. 2 is a schematic block diagram illustrating the system architecture of the perioperative cobotic platform 100 and its major functional modules. At the core of the system is a control unit 202 (one or more processors with associated memory and hardware interfaces) that executes the software modules and coordinates all sensors and actuators. The multi-modal imaging suite 210 (corresponding to the physical sensors 112, 114, 116, 118 on the cobot) provides raw input data streams including video, depth maps, and thermal readings. These sensor feeds are delivered to a sensor fusion and environment mapping module 220, which combines the data into a unified representation of the operating room environment. Module 220 continuously updates a dynamic 3D map of the OR, identifying objects and surfaces and tracking changes (as will be described in reference to FIG. 4).

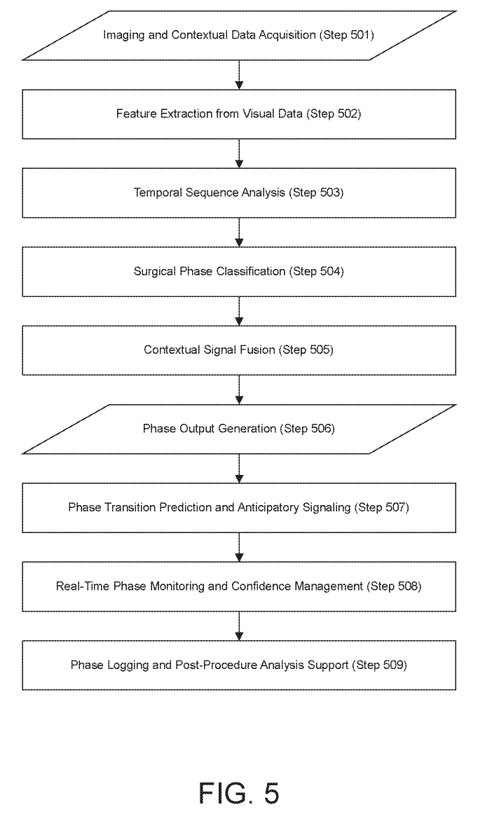

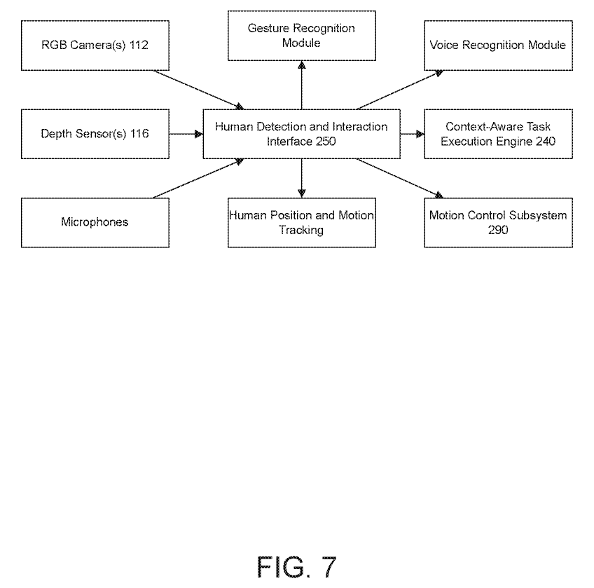

In parallel with mapping, the system runs a surgical context recognition pipeline. A surgical phase detection engine 230 analyzes visual and other sensor cues to determine the current phase of the procedure (see FIG. 5 for details). Similarly, a human detection and interaction interface module 250 processes sensor data (such as camera feeds and depth data) to recognize the presence, positions, and gestures or voice commands of surgical team members (as described in FIG. 7). The system architecture optionally includes audio input devices (microphones) connected to module 250 for speech recognition, enabling surgeon spoken commands to be captured and interpreted. Outputs from modules 230 (phase) and 250 (human/commands) are provided to a context-aware task execution engine 240.

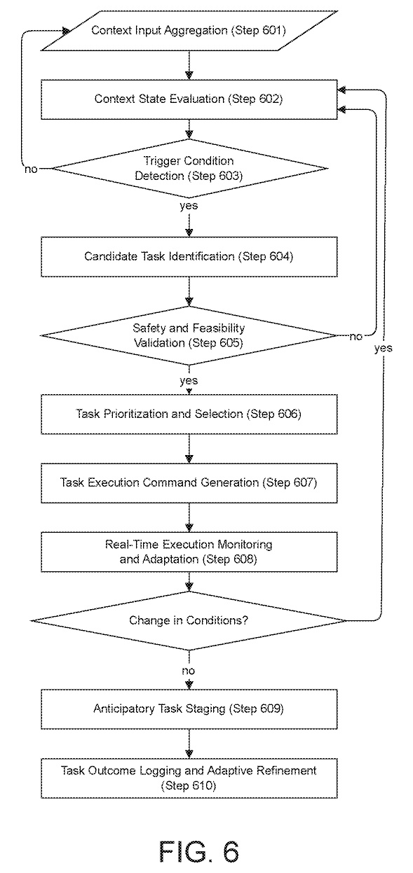

The context-aware task execution engine 240 is responsible for deciding and initiating cobot actions in response to the recognized surgical context. Engine 240 receives inputs from the mapping module 220 (for situational awareness), the phase detection engine 230 (for procedural context), and the human interaction module 250 (for commands and proxemics). Based on these inputs, engine 240 triggers appropriate behaviors or tasks for the cobot, such as moving the base, repositioning the arm, or operating a tool. It may utilize a knowledge base of surgical workflows and a set of pre-defined or learned rules so that actions are executed at contextually appropriate times (see FIG. 6 for examples of task logic). Commands from engine 240 are passed to the cobot’s motion control subsystem 290, which encompasses the low-level controllers for the drive base and arm motors. The motion control subsystem 290 converts high-level movement or action commands into specific actuator commands, executing tasks while ensuring smooth and safe operation.

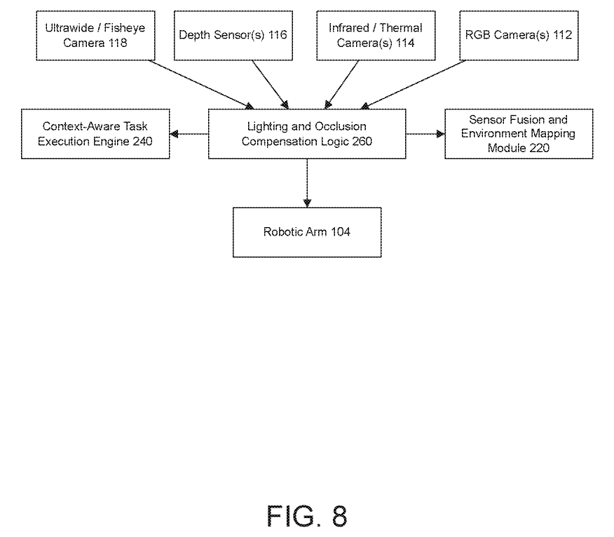

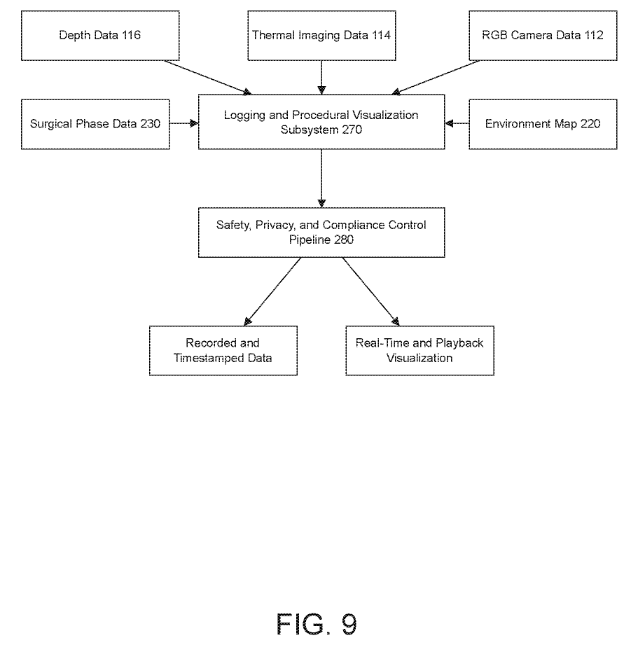

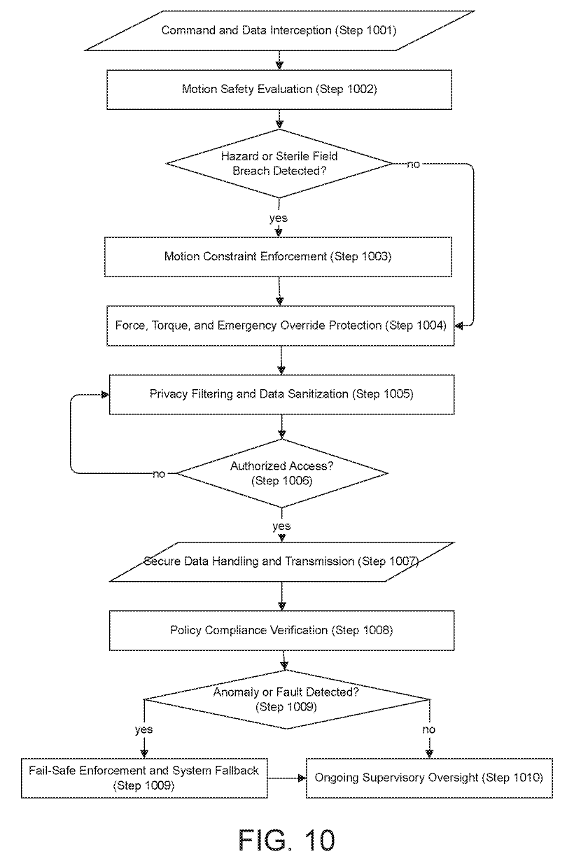

Additional supporting modules interact with this core data flow. A lighting and occlusion compensation logic module 260 monitors the quality of sensor inputs and external illumination conditions, and can adjust camera settings, activate auxiliary lighting, or suggest slight repositioning of the cobot (via engine 240) to mitigate any loss of visual data (see FIG. 8). A logging and procedural visualization subsystem 270 records key data streams and events to memory or secure storage. Subsystem 270 can also generate a live visualization of the cobot’s perspective or the real-time OR map, which can be displayed on a user interface (such as a tablet, monitor, or AR headset) either in the OR or remotely (as described in FIG. 9). Finally, a safety, privacy, and compliance control pipeline 280 permeates the system. This module 280 enforces safety constraints (e.g., collision avoidance and sterile field boundary limits) on the motion commands issued by subsystem 290, and it filters or restricts data outputs from subsystem 270 to comply with privacy and hospital policies (detailed in FIG. 10). Module 280 may halt or override any action that would violate safety parameters, and it ensures all recorded or transmitted data is appropriately anonymized or access-controlled.

Overall, FIG. 2 shows that the system architecture is highly modular: the multi-modal sensors 210 feed into perception modules (220, 230, 250), which inform the decision module 240, which in turn drives the robotic hardware 290 to act. Throughout this process, the lighting/occlusion logic 260 continuously optimizes sensor performance, the logging subsystem 270 archives data and provides situational awareness to humans, and the safety/privacy controls 280 serve as a gating layer to guarantee safe, compliant operation at all times. The control unit 202 may run these modules concurrently with real-time scheduling, ensuring that critical tasks (like obstacle detection and motion control) have priority to meet real-time safety requirements, while higher-level analyses (like phase detection) run in parallel threads. In some embodiments, the computing resources for these modules are distributed: for example, heavy image processing or machine learning inference (for phase or gesture recognition) might be offloaded to a GPU or an edge server connected via a high-speed link, whereas motion control loops run on a local microcontroller. Network interfaces may connect the system to hospital information systems (for pulling case-specific data or pushing logged data) provided that such connections meet the required security and latency criteria.

FIG. 3 provides an overview of the multi-modal imaging suite 110 of the cobot platform. The suite comprises multiple sensor types that each contribute distinct information about the operating room environment. By combining these modalities, the cobot achieves robust perception under a variety of conditions. In this embodiment, the imaging suite 110 includes at least the following components:

RGB Optical Camera(s) 112: High-resolution color video cameras that capture visual details of the scene. These provide real-time images of surgical staff, the patient, instruments, and room objects. They are used for tasks like recognizing personnel and tools by appearance and monitoring procedural steps. Multiple cameras 112 may be placed around the cobot (for example, one on the robotic arm focusing on the surgical field and additional units on the base for peripheral vision) to cover different angles without repositioning the robot.

Infrared/Thermal Imager(s) 114: One or more infrared cameras sensitive to thermal radiation, used to detect heat signatures. Thermal imagers 114 can identify people by body heat, even in low ambient light or if they are partially obscured from view of the optical cameras. They also help monitor equipment status (for instance, identifying an overheating device or confirming that warming blankets or IV fluids are at proper temperatures). IR imaging complements visible-light cameras by remaining effective during lighting changes (such as when surgical lights are dimmed) and by seeing through certain visual obstructions like smoke.

Depth Sensor(s) 116: Sensors that measure distance to surfaces and objects, providing a 3D point cloud or depth map of the environment. Examples include LiDAR units emitting laser scans around the room or stereo camera systems computing depth via parallax. Depth sensors 116 allow the cobot to perceive the spatial layout of the OR, including the positions of walls, furniture, equipment, and people. This real-time 3D data is crucial for safe navigation and for the sensor fusion module to construct an accurate spatial map. The depth sensor(s) typically have a range and resolution sufficient to detect both large structures (e.g., an OR table or overhead lights) and finer details (e.g., an outstretched arm or a small cart) within the cobot’s vicinity.

Ultrawide/Fisheye Camera(s) 118: One or more wide-angle imaging devices are included to provide a broad field of view covering nearly the entire room from a single vantage point. For instance, a fisheye camera 118 mounted on top of the cobot can capture a 360-degree panoramic view or a very wide-angle view that eliminates blind spots directly behind or beside the robot. This ensures that the system is aware of approaching staff or moving equipment even outside the main field of view of the narrow-focus cameras. Ultrawide images may be de-warped and used in conjunction with the other camera feeds to maintain continuous situational awareness.

Each of these sensors 112, 114, 116, 118 is calibrated with respect to the cobot’s frame of reference so that their data can be correlated. For example, the optical and depth sensors may be calibrated for intrinsic parameters and extrinsic positions such that a point detected in 3D by a LiDAR 116 can be accurately overlaid on the camera image 112. The sensors are also time-synchronized (using hardware timestamps or an internal clock) to facilitate sensor fusion. Data from all modalities is streamed concurrently to the control unit 202. In some configurations, additional specialized sensors might be part of suite 110, such as a microphone array for sound localization, or even ultraviolet or fluorescence cameras to detect specific materials. However, the core set listed above provides comprehensive environmental imaging without relying on any invasive or ionizing radiation modalities.

FIG. 4 is a diagram illustrating the operation of the sensor fusion and spatial mapping module 220. In this process, raw data from the various imaging modalities (cameras 112, depth sensor 116, IR sensor 114, etc.) are collected and co-registered in a common spatial frame. The fusion module 220 aligns the data both spatially and temporally: for instance, it associates depth points from sensor 116 with pixels from camera 112 to colorize a point cloud of the environment, and correlates thermal readings from IR camera 114 with corresponding locations. Using techniques such as simultaneous localization and mapping (SLAM) or extended Kalman filter-based sensor fusion, module 220 integrates successive sensor readings to build and maintain a dynamic three-dimensional map of the operating room.

The output of module 220 is a unified spatial model that represents all major structures, objects, and agents in the OR. This model can be conceptualized as an occupancy grid or a 3D mesh/point cloud in which each cell or point is annotated with information (such as whether it is free space, part of a wall or equipment, or currently occupied by a person). Static fixtures like walls, the ceiling, large equipment consoles, or the operating table 152 are mapped and remain in a fixed position in the model. Movable objects and dynamic obstacles are also captured: for example, if a nurse moves an instrument cart or if a monitor is repositioned, the sensor fusion module detects the change by comparing new depth data against the prior map and updates the representation accordingly. Team members (surgeon, nurses) appear in the map as moving entities; their positions are continually updated as they walk or reach into different areas, leveraging both the depth data and the human recognition cues from module 250.

Importantly, the sensor fusion algorithm distinguishes between static and dynamic elements. By analyzing motion over time (e.g., tracking clusters of depth points or visual features frame-to-frame), the system can label regions of the map that correspond to moving persons or objects versus those that are stationary. This allows other modules to treat them appropriately, for instance, the path planning logic (within motion control 290) knows to anticipate that a dynamic obstacle might move out of the way, whereas a static obstacle must be navigated around. The mapping module 220 also incorporates known reference information if available: for example, the approximate location of the operating table 152 or ceiling-mounted lights might be preloaded or recognized by markers, helping to initialize the map and to define no-go zones like the patient area.

One specific layer of the spatial model is the identification of the sterile field boundary 154. Module 220 can infer the sterile zone by detecting the positions of surgical drapes (for instance, a large rectangular region around patient 150 at table height might be recognized via color segmentation of blue sterile drape material in the camera images). Alternatively or additionally, the sterile field region can be defined in advance (e.g., input from staff or from integration with the OR setup data) and then projected onto the map so that the system is constantly aware of this boundary. This information is fed forward to the safety/compliance module 280 to ensure the cobot does not inadvertently breach sterile areas. Likewise, the fusion module flags any intrusion into the sterile field by unanticipated objects (like a non-sterile item or a person’s un-gowned arm) by combining visual cues and spatial location, which can trigger an alert.

The sensor fusion and mapping module 220 runs continuously throughout the procedure, updating the environment model in real time (for example, on the order of many times per second) as new sensor data arrives. It outputs updated spatial information to other subsystems. For instance, context engine 240 queries the map to find free space for the cobot to move or to locate where a needed instrument cart is currently positioned. The mapping module also provides the robot’s own location within the map frame (by matching features from sensors to the map, the cobot localizes itself in the room). In some embodiments, the map-building is robust against temporary occlusions: if one camera view is blocked, the module can rely on other sensors or recent data to avoid immediately dropping objects from the model. As a result, module 220 yields a stable, comprehensive awareness of the environment, enabling safe navigation and informed decision-making by the cobot.

FIG. 5 depicts the operation of the surgical phase detection engine 230, which uses machine learning and pattern recognition to classify a current phase of a surgical procedure in real time. The process begins with imaging and contextual data acquisition (step 501), in which the phase detection engine 230 receives input primarily from the imaging suite 110, including optical camera feeds 112 capturing the surgical field, personnel movements, and instrument activity. In some embodiments, additional contextual signals are also received, such as timing information, instrument usage data, activation states of surgical devices, or outputs from the environment mapping module 220.

The process continues with feature extraction from visual data (step 502), wherein individual video frames or short frame sequences are processed by one or more trained feature extraction models. In one embodiment, a deep convolutional neural network (CNN) analyzes each frame to identify salient visual features, such as the presence and type of surgical instruments, positions and motions of the surgeon’s hands, changes in tissue appearance, or spatial relationships between tools and the surgical site.

Next, the extracted features are evaluated using temporal sequence analysis (step 503) to determine how visual patterns evolve over time. In some embodiments, a temporal model, such as a long short-term memory (LSTM) recurrent neural network, gated recurrent unit (GRU), or Hidden Markov Model, processes the sequence of extracted features to recognize transitions and temporal signatures corresponding to known surgical phases.

Based on the temporal analysis, the engine performs surgical phase classification (step 504), in which the current procedural phase is inferred. By way of example, identifiable phases may include a setup phase (patient positioning and sterile preparation), an incision phase, a primary operative or resection phase, and a closing phase involving suturing or dressing application. The phase detection engine 230 may be trained using annotated datasets of prior surgical procedures, enabling the model to learn visual and temporal cues associated with phase transitions.

In some embodiments, the phase classification is further refined through contextual signal fusion (step 505), in which non-visual inputs are incorporated to improve accuracy. For example, detection of electrocautery activation, recognition of a specific instrument handoff, identification of a surgical robot entering an active state, or spatial changes detected by the environment mapping module 220 may be used as corroborating evidence to confirm or adjust the inferred phase.

Once a phase determination is made, the engine performs phase output generation (step 506), outputting a discrete phase label or a probability distribution over multiple candidate phases to the context-aware task execution engine 240. This output enables the cobot to adapt its behavior in alignment with the current procedural context. Certain cobot actions may be enabled, disabled, or prioritized based on the identified phase.

In some embodiments, the process further includes phase transition prediction and anticipatory signaling (step 507), wherein the phase detection engine 230 detects patterns indicative of an impending phase transition. Based on this prediction, the context-aware task execution engine 240 may preemptively reposition the cobot, prepare tools, or adjust system configurations in anticipation of the next phase, thereby reducing workflow latency.

The phase detection engine 230 operates continuously through real-time phase monitoring and confidence management (step 508), updating its assessment as new imaging data is received. If classification confidence is below a threshold, the engine may maintain the previously identified phase to avoid rapid oscillation, or may output a confidence value or ambiguity flag for optional human verification.

Finally, the system performs phase logging and post-procedure analysis support (step 509), in which detected phases, confidence levels, and timestamps are recorded by the logging and procedural visualization subsystem 270. Logged phase data may be used for postoperative review, performance analysis, training, or iterative refinement of the machine learning models.

FIG. 6 illustrates the operation of the context-aware task execution engine 240, which orchestrates the cobot’s supportive actions during surgery based on real-time contextual information. The process begins with context input aggregation (step 601), in which the task execution engine 240 receives interpreted situational data from multiple subsystems, including the sensor fusion and environment mapping module 220, the surgical phase detection engine 230, and the human detection and interaction interface 250.

The engine then performs context state evaluation (step 602), wherein the current operative context is assessed. This evaluation may include determining the current surgical phase, identifying locations and activities of surgical team members, detecting direct verbal or gestural commands, and assessing environmental conditions within the operating room, such as equipment placement, sterile field boundaries, and available free space.

Based on the evaluated context, the engine performs trigger condition detection (step 603) to determine whether one or more predefined conditions for cobot assistance have been satisfied. Trigger conditions may be defined by rule-based logic, hierarchical state machines, or machine-learning-based decision policies trained on prior procedures. The engine continuously monitors for such triggers while maintaining an unobtrusive operational posture.

When a trigger condition is detected, the engine proceeds to candidate task identification (step 604), in which one or more potential cobot actions associated with the detected context are identified from a task library. Example candidate tasks may include instrument delivery, suction activation, drape adjustment, lighting adjustment, camera positioning, or cobot repositioning for improved line-of-sight.

The identified candidate tasks are then subjected to safety and feasibility validation (step 605), in which each proposed action is evaluated for safety, sterility compliance, and interference avoidance. This validation may include checking proximity to personnel, ensuring compliance with sterile field constraints, and obtaining clearance from the safety, privacy, and compliance control pipeline 280 before any physical motion is initiated.

Upon validation, the engine performs task prioritization and selection (step 606), ranking candidate actions based on urgency, relevance to the current surgical phase, and contribution to workflow efficiency. Lower-priority or redundant tasks may be suppressed, for example when a human team member is already performing the same function or when the task would be disruptive during a critical operative moment.

Once a task is selected, the engine initiates task execution command generation (step 607), issuing control instructions to the motion control subsystem 290 to carry out the selected action. For example, this may include commanding the robotic arm 104 to present an instrument, reposition a suction device, adjust a drape or light, or directing the mobile base 102 to move to a new vantage point determined using updated map data from module 220.

During execution, the system performs real-time execution monitoring and adaptation (step 608), continuously monitoring sensor feedback to ensure the task proceeds safely and as intended. If conditions change mid-task, such as a person entering the cobot’s path, the surgeon aborting a maneuver, or a new obstacle appearing, the engine may pause, modify, or abort the task and optionally re-evaluate context conditions.

In parallel or subsequent to execution, the engine may perform anticipatory task staging (step 609), wherein upcoming tasks are prepared based on predicted workflow progression. For example, the cobot may pre-position an instrument or adjust its configuration in anticipation of a forthcoming surgical phase identified by the phase detection engine 230, while delaying final execution until an explicit readiness cue is detected.

Finally, the process includes task outcome logging and adaptive refinement (step 610), in which executed actions, timing data, and contextual conditions are recorded by the logging and procedural visualization subsystem 270. In some embodiments, this information is used to adapt task timing, trigger sensitivity, or prioritization rules for future procedures, enabling the task execution engine 240 to refine its behavior over time and better align with surgeon-specific workflows.

FIG. 7 illustrates the human detection and interaction interface module 250, which enables the cobot to collaborate smoothly with surgical staff. This module encompasses the algorithms and sensors used to recognize people in the environment and interpret their deliberate signals (gestures or voice commands) while also tracking their positions for safety. The cobot’s vision system (cameras 112 and depth sensors 116) detects human figures and uses techniques such as pose estimation or skeletal tracking to understand body positions and motions. It can distinguish key individuals in the OR, for example, detecting which person is the lead surgeon versus support staff, based on their location relative to the patient or via pre-configuration (e.g., the surgeon might wear a distinctive marker or simply be the person nearest the operating table head). The system continuously updates each detected person’s location and movement trajectory so that the cobot is aware of where people are and where they are likely to move next.

Using this information, the cobot adjusts its own actions to be non-disruptive. For instance, module 250 informs the motion control system 290 of human positions, enabling real-time collision avoidance and fluid navigation around people. If a staff member comes too close to the moving base 102 or arm 104, the cobot automatically slows or pauses to avoid any contact. It also avoids blocking humans’ paths or important sight lines: for example, the cobot will not position itself between the surgeon and the patient or stand in front of a vital sign monitor that staff need to see. By recognizing typical human motion patterns (like a nurse frequently moving between the instrument table and patient), the interface 250 can predict those trajectories and keep those pathways clear.

In addition to passive tracking, the human interaction interface allows direct communication from the team to the robot. The system supports hands-free control through simple gestures and voice commands so that sterile technique is maintained (no need to touch any device). One or more microphones on the cobot capture spoken commands; the audio is processed by speech recognition software (which may be specialized for the OR context to filter out background noise and respond to known command phrases). For example, a surgeon could say, “cobot, come here,” or “increase zoom,” and the system will interpret and execute the command. Gesture recognition works via the vision sensors: the module 250 can identify gestures such as a wave to get the robot’s attention, a pointing gesture toward an object (indicating, perhaps, that the robot should focus or move something there), or a hand signal like a raised open palm meaning “stop”. All such inputs are relayed to the context engine 240, which then decides on the appropriate response (as discussed earlier, e.g., delivering a tool or pausing movement).

To facilitate this interaction, the cobot provides feedback to the humans. Module 250 may control indicator lights or a speaker on the cobot to acknowledge commands (for instance, a brief chime or LED flash when it recognizes a voice command, or a verbal confirmation like “Understood”). In some embodiments, the cobot includes a small display or projects an icon to signal its current mode or to whom it is attending (for example, it might show an icon indicating it is in ‘listening’ mode when a person says the wake word). The system is also capable of learning user preferences, if one surgeon prefers gesture controls while another uses voice, the interface can adapt by prioritizing the more commonly used modality for that user, possibly identified by voice ID or prior configuration.

Overall, the human detection and interaction interface 250 ensures that the cobot integrates into the surgical team as a cooperative presence. It actively monitors human positions to maintain safety and uses intuitive interaction methods to receive instructions without requiring physical contact or complex controllers. By understanding human cues and providing immediate feedback, the system builds trust and situational fluency, team members can work around the cobot confidently, and when they need its assistance or to modify its behavior, they can do so naturally with a word or gesture.

FIG. 8 shows the lighting and occlusion compensation logic 260, which helps the cobot maintain reliable vision and mapping despite changes in operating room lighting or temporary obstructions of sensors. The OR environment can vary from brightly lit (during open surgery with overhead surgical lights) to dim or near-dark (during endoscopic or arthroscopic procedures where room lights are turned off to enhance monitors). Additionally, moving personnel or equipment can sometimes block a camera’s view. Module 260 monitors the quality and status of the incoming sensor data and dynamically adjusts the system to compensate for such conditions.

For lighting variations, the logic 260 can automatically tune the imaging parameters of the cameras 112. If the ambient light drops, the system may increase camera gain or exposure time, or switch to a camera with better low-light sensitivity (such as an IR-enhanced camera). Conversely, if a light is suddenly switched on causing glare or overexposure, the module can reduce exposure or engage high-dynamic-range (HDR) imaging modes. In one embodiment, the cobot carries its own auxiliary illumination: for example, an LED light ring around a camera that it can turn on to illuminate a region of interest with controlled light (possibly using wavelengths that do not interfere with surgeons’ vision, like infrared). This ensures that the cobot’s vision is not completely dependent on external lighting. The compensation logic also works to maintain color fidelity and consistency of the visual feeds so that recognition algorithms (like phase detection or object tracking) remain effective under different lighting.

Occlusion handling is another critical aspect. The system uses overlapping fields of view from multiple sensors so that if one camera’s view is blocked, another sensor can still provide data. For instance, if a staff member stands in front of the main RGB camera 112, the cobot might still track positions using the depth sensor 116 from a slightly different angle or rely on the fisheye camera 118 that sees around the obstruction. The logic 260 can also prompt the cobot to subtly reposition to regain a lost line of sight: it might raise or rotate the arm 104 holding a camera to peek over the obstacle or shift the base a small distance to get an alternate view, assuming such movement does not disrupt the procedure. All these adjustments are computed so as to not surprise or interfere with the staff, movements are slow and announced via the interface if needed (e.g., a gentle audio cue that the cobot is adjusting for visibility).

The multi-modal nature of the imaging suite inherently lends redundancy. If heavy smoke from cauterization obscures optical views, the thermal camera 114 can still detect heat signatures of the patient and instruments, helping the system maintain awareness until the smoke clears. Likewise, if the IR camera’s data is saturated or if bright surgical lights wash out thermal contrast, the high-resolution optical camera or depth sensor can compensate. Module 260 continuously evaluates metrics like image contrast, number of tracked feature points, and depth return quality. When these metrics fall below thresholds, it triggers the appropriate response, whether that’s switching sensor priority, adjusting settings, or repositioning.

By employing the lighting and occlusion compensation logic 260, the cobot ensures that critical functions like localization (module 220) and object detection remain uninterrupted. This contributes to overall safety; for example, the cobot will not make a blind movement if its view is compromised. Instead, it can either pause motion until visibility is restored or rely on the last known map with caution. In summary, module 260 makes the vision system robust to real-world operating room dynamics, maintaining visual continuity and confidence in the sensor data despite transient environmental changes.

FIG. 9 illustrates the logging and procedural visualization subsystem 270, which handles data recording and real-time display of information from the cobot. This subsystem operates in the background throughout the surgical procedure, capturing a synchronized record of critical data streams: video from each camera 112, depth maps from sensor 116, thermal imagery 114, as well as high-level events such as detected surgical phase changes (from engine 230) and cobot actions or alerts. All data are timestamped and stored in a secure data repository, either on the cobot’s onboard storage or on a connected hospital server. The recorded dataset forms a comprehensive “black box” of the surgery, useful for later analysis, training, and compliance.

During the operation, subsystem 270 can provide live visualization on one or more displays to enhance situational awareness. For example, a monitor in the OR or a tablet held by a circulating nurse could show the cobot’s perspective camera feed with annotations. Annotations might include outlines or highlights around detected team members or tools, the current identified surgical phase, and perhaps the cobot’s intended next action (so the team knows what the robot is about to do). Another view can show a top-down map of the OR (from the data in module 220), indicating the real-time positions of the cobot and staff as colored icons, along with zones like the sterile field boundary 154 clearly marked. Such visualizations let the surgical team or observers quickly grasp what the cobot “sees” and plans, building trust and enabling easy supervision. Remote viewers (for instance, a teaching proctor in another room or a command center) can also access these live feeds securely to monitor progress without being physically present.

After the procedure, the logged data is available for review and analysis. The system can automatically compile a procedural timeline using the phase detection log: e.g., noting the start and end times of each phase and any significant events. Surgeons and quality improvement personnel can replay synchronized video from different angles alongside this timeline to identify any inefficiencies or training opportunities. For instance, the log might show that a certain phase took longer than usual, prompting a review of the footage to see if there were unanticipated delays or equipment searches. Instrument usage can also be extracted (if the cobot tracks which instruments were handed over and when), providing metrics like how many times each tool was used or how long it was held, which are valuable for inventory management and surgeon feedback.

The data logging is done in a manner compliant with privacy and security standards (see FIG. 10): patient identifying information is either not recorded or is protected. Access to the recorded data is role-based; for example, surgeons can review their own cases for learning, but only authorized quality officers might access full archives, and any shared videos for education would have patient faces or sensitive areas blurred out by default. Additionally, the system’s machine learning components can utilize logged data to improve over time (with appropriate anonymization), for instance, difficult or novel scenarios captured in the logs can be added to the training set to refine the phase detection or context decision models. Thus, subsystem 270 not only provides immediate benefits in visualization and transparency but also creates a feedback loop for continual system improvement and accountability.

FIG. 10 depicts the operation of the safety, privacy, and compliance control pipeline 280, which envelops the cobot’s operations with protective measures to ensure patient safety, data security, and adherence to applicable medical regulations. The process begins with command and data interception (step 1001), in which the pipeline 280 operates as a parallel monitoring layer that receives motion commands, control instructions, sensor data, and data transmission requests before such commands are executed or such data is stored or transmitted.

The pipeline next performs motion safety evaluation (step 1002), wherein all movement commands generated by the context-aware task execution engine 240 or by any direct or remote control input are vetted for safety. This evaluation may include applying collision avoidance algorithms that compare planned motion paths against the current environment map generated by the sensor fusion and environment mapping module 220, ensuring that no collisions are predicted within a defined safety margin.

If potential hazards are detected, the pipeline performs motion constraint enforcement (step 1003), in which movement parameters are modified or restricted. This may include adjusting a planned path, reducing speed or acceleration, or halting motion entirely and issuing a warning. The pipeline enforces defined keep-out zones corresponding to sensitive regions, including the sterile field 154 and the patient’s body, ensuring that the mobile base 102 does not enter such zones and that the robotic arm 104 enters only when explicitly authorized and appropriately draped.

The system further applies force, torque, and emergency override protection (step 1004), imposing limits on velocity, acceleration, and applied force regardless of commanded values. Joint torque sensors and force-limited actuators monitor for unexpected resistance indicative of human contact or obstruction, triggering an immediate stop when thresholds are exceeded. In addition, hardware emergency stop mechanisms, including a physical E-stop on the cobot and a wireless kill-switch accessible to staff, provide a manual override that instantaneously disables motion.

In parallel with motion safety, the pipeline performs privacy filtering and data sanitization (step 1005) before any imaging or sensor data is logged or transmitted. This may include automatically blurring or obscuring faces of patients and staff, suppressing audio recording to avoid capturing sensitive conversations, or selectively disabling capture of restricted views such as patient faces or computer screens displaying patient records.

The pipeline then performs access control and authorization verification (step 1006), in which requests to view, store, or transmit data are checked against authenticated user identities and role-based access permissions. Full-resolution surgical data may be made available only to authorized clinicians, while data shared for training, auditing, or remote consultation may be restricted or anonymized in accordance with institutional policy.

Next, the system executes secure data handling and transmission (step 1007), ensuring that all stored data is encrypted and tagged with access control metadata, and that any data transmitted off the cobot, such as to a hospital server or remote viewing device, is sent over secure communication channels meeting regulatory requirements, including encryption in transit and authenticated endpoints consistent with HIPAA guidelines.

The pipeline further includes policy compliance verification (step 1008), in which hospital-specific rules and regulatory constraints are enforced. For example, recording may be disabled until explicit consent is confirmed through a digital approval mechanism, or certain imaging modes may be prohibited entirely based on institutional policy. The pipeline enforces compliance with applicable medical device, robotics, and cybersecurity standards, including IEC 60601, ISO 10218, and FDA cybersecurity guidance, as applicable.

If any anomaly, uncertainty, or internal fault is detected, the pipeline performs fail-safe enforcement and system fallback (step 1009), transitioning the cobot into a safe state. This may include pausing motion, disabling autonomous actions, securing data, and awaiting human intervention rather than allowing continued operation under uncertain conditions.

Through continuous execution of these steps, the safety, privacy, and compliance control pipeline 280 performs ongoing supervisory oversight (step 1010), ensuring that all cobot actions and data processes remain deliberate, safe, and compliant throughout the surgical procedure. This layered oversight enables advanced autonomous operation while maintaining the high standards of safety, confidentiality, and regulatory adherence required in perioperative environments.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A perioperative cobotic system with multiple imaging modalities for environmental awareness, comprising:
a mobile base configured for autonomous navigation within an operating room, including obstacle avoidance and sterile-field compliance;
a multi-modal imaging suite carried by the mobile base, the imaging suite comprising a plurality of imaging devices including at least one visible-light camera, at least one depth sensor, at least one wide-angle fisheye camera, and at least one infrared camera, the imaging suite being arranged to capture real-time visual and spatial data of the surgical environment;
a sensor fusion and environment mapping module operatively coupled to the imaging suite and configured to combine outputs from the imaging devices to generate a unified, real-time three-dimensional map of the operating room, including locations of equipment, personnel, and defined sterile zones, and to continuously update the map as conditions change;
a context-aware task execution control system communicatively connected to the sensor fusion module and configured to autonomously trigger and adjust cobot actions in alignment with detected surgical workflow events and environmental conditions;
a surgical phase detection engine comprising at least one machine learning model trained to recognize visual patterns indicative of procedural phases, the engine being configured to analyze imaging data from the imaging suite and infer a current phase of a surgical procedure, and to cause the context-aware task execution control system to perform one or more predefined cobot actions associated with the inferred phase; and
a collaborative human detection and interaction interface comprising sensors and processing components configured to identify locations and gestures or voice commands of surgical team members, enabling the cobot to operate safely alongside personnel and to respond to surgeon inputs in an intuitive manner.

2. The system of claim 1, wherein the multi-modal imaging suite includes at least one panoramic fisheye camera mounted on the cobot to provide a substantially 360-degree view of the operating room, thereby minimizing blind spots in the cobot’s visual coverage.

3. The system of claim 1, further comprising a calibration module configured to periodically calibrate the imaging devices of the multi-modal imaging suite to a common reference frame, ensuring accurate sensor fusion and alignment of the multi-modal data.

4. The system of claim 1, wherein the sensor fusion and environment mapping module is further configured to identify sterile field boundaries within the operating room and mark those boundaries on the real-time map, and wherein the context-aware task execution control system automatically restricts the cobot’s movement to prevent the cobot from breaching the marked sterile field boundaries during operation.

5. The system of claim 1, wherein the context-aware task execution control system is configured to recognize predefined events in the surgical workflow and respond by performing corresponding assistance tasks, including at least one of delivering a surgical instrument to a team member, adjusting an overhead surgical light, or repositioning a suction device, when such actions are contextually appropriate.

6. The system of claim 1, wherein the context-aware task execution control system is further configured to predict a forthcoming phase of the surgical procedure based on the current phase and ongoing activities, and to preemptively reposition the cobot or adjust the cobot’s configuration in anticipation of the forthcoming phase.

7. The system of claim 1, wherein the surgical phase detection engine comprises a machine learning model trained on a dataset of surgical procedures annotated with corresponding procedural phases, the engine being thereby configured to improve phase inference accuracy by applying patterns learned from the annotated surgical data.

8. The system of claim 1, wherein the collaborative human detection and interaction interface includes a gesture recognition module configured to interpret predetermined hand gestures made by a surgeon as commands to control or adjust the cobot’s actions.

9. The system of claim 1, wherein the collaborative human detection and interaction interface includes a voice recognition module configured to detect and interpret verbal commands from authorized surgical staff to adjust the cobot’s operation accordingly.

10. The system of claim 1, further comprising a logging and visualization module configured to record synchronized multi-modal imaging data and cobot action data throughout a surgical procedure and to provide real-time or playback visualizations of the cobot’s perspective and the environment map on one or more displays for analysis, training, or review purposes.

11. A perioperative cobotic system with multiple imaging modalities for environmental awareness, comprising:
a mobile base configured for autonomous movement and navigation in a surgical environment;
a multi-modal imaging suite comprising at least one optical camera and at least one depth sensor to capture real-time data of the operating room;
a control system operatively coupled to the imaging suite and configured to fuse the captured data, identify a current surgical phase using a machine learning algorithm, and execute context-aware tasks in coordination with the surgical workflow;
a communication interface configured to transmit live imaging and mapping data from the cobot to one or more remote display devices and to receive external data from hospital information systems; and
one or more privacy protection mechanisms configured to filter or obscure sensitive patient information in the transmitted data and to enforce access controls in compliance with patient privacy regulations.

12. The system of claim 11, wherein the communication interface is configured to provide a live video feed of the cobot’s camera views and the real-time environmental map to one or more remote computing devices to enable remote monitoring or consultation during the surgical procedure.

13. The system of claim 11, wherein all data transmitted via the communication interface is protected by end-to-end encryption to meet security and confidentiality requirements for patient information.

14. The system of claim 11, wherein the cobot requires authentication of remote users and implements role-based access control such that only authorized medical personnel can view live data or issue commands through the communication interface.

15. The system of claim 11, wherein the communication interface is configured to retrieve patient-specific or procedure-specific information from a hospital information system and automatically adjust the cobot’s operational settings based on the retrieved information.

16. The system of claim 11, wherein the communication interface enables an authorized remote user to send control instructions to the cobot, including commands to adjust the cobot’s camera viewpoint or position, and wherein the control system executes any such remote commands while maintaining local safety constraints to prevent interference with the surgical team.

17. A computer-implemented method for context-aware operation of a perioperative cobotic system, the method comprising:
collecting imaging data from a plurality of imaging devices of a multi-modal imaging suite during a surgical procedure;
analyzing the collected imaging data using a trained machine learning model to identify a current phase of the surgical procedure based on visual patterns present in the data;
determining one or more context-specific actions for the cobot to perform, the actions being selected based on the identified surgical phase; and
triggering the cobot to perform the one or more determined actions in coordination with the surgical workflow corresponding to the identified phase.

18. The method of claim 17, wherein analyzing the imaging data to identify the current surgical phase comprises inputting the imaging data into a neural network model trained on a plurality of recorded surgical procedures annotated with phase labels, such that the model outputs an indication of the current procedural phase based on learned patterns in the data.

19. The method of claim 17, further comprising predicting a subsequent surgical phase based on the identified current phase and recent observed trends in the imaging data, and preemptively adjusting a position or operational configuration of the cobot in preparation for the predicted subsequent phase.

20. The method of claim 17, wherein identifying the current surgical phase further includes detecting contextual cues from the imaging data including one or more of: a presence or usage of particular surgical instruments, positions or movements of surgical personnel, or changes in the surgical field, and using those cues as additional inputs to improve the accuracy of the phase determination.
